(19) United States
(12) Patent Application Publication
(10) Pub. No.: US 2016/0000873 A1
(43) Pub. Date: Jan. 7, 2016

(54) AGENTS WHICH INDUCE LYMPHANGIOGENESIS FOR USE IN THE TREATMENT OF CYSTIC KIDNEY DISEASE

(71) Applicant: UCL BUSINESS PLC, London, Greater London (GB)

(72) Inventors: David LONG, London (GB); Paul WINYARD, London (GB); Jennifer HUANG, London (GB)

(21) Appl. No.: 14/765,133

(22) PCT Filed: Feb. 14, 2014
(86) PCT No.: PCT/GB2014/050436

§ 371 Date: Jul. 31, 2015

Related U.S. Application Data
(60) Provisional application No. 61/764,626, filed on Feb. 14, 2013.

(51) Int. Cl. A61K 38/18; A61K 48/00; A61K 31/7088; A61K 9/00; A61K 45/06; C07K 14/475; A61K 38/17

(52) U.S. Cl. CPC A61K 38/1866; C07K 14/475; A61K 48/005; A61K 38/1891; A61K 38/1783; A61K 38/179; A61K 9/0019; A61K 45/06; A61K 31/7088

(57) ABSTRACT

The invention relates to methods and materials for treating a renal cystic disease in a subject suffering therefrom, the methods comprising administering the compound to the subject, wherein the compound is a lymphangiogenic agent such as an agonist of VEGFR-3, or a nucleic acid encoding said agent.

### TECHNICAL FIELD

The present invention relates generally to methods and materials for use in treating cystic kidney diseases, particularly polycystic kidney disease.

### BACKGROUND ART

Polycystic kidney disease (PKD) causes morbidity, renal failure and death from before birth through adulthood.

PKD is characterised by the growth of multiple fluid-filled cysts leading to a loss of normal kidney structure and functions that in many cases result in end-stage renal disease.

The recessively inherited form (ARPKD) occurs in 1 in 20,000 and predominantly affects children who can present at any stage from prenatally through adolescence; nearly all will develop renal failure and need dialysis and/or transplantation.

The dominant form is more common at 1 in 600, and causes kidney failure in around 50% of cases, usually around middle age. ADPKD accounts for 2-3% of adult dialysis programme patients, which equates to a significant health care cost (Lentine K L et al Clin J Am Soc Nephrol 2010 5:1471-1479)

To-date, most treatment strategies have targeted disrupted cellular functions within the cysts themselves but this approach has yet to generate clinically approved therapies for PKD.

In particular, strategies have been trialled to reduce cyst development but most appear ineffective at tolerable doses in humans apart from Tolvaptan, a selective competitive vasopressin receptor 2 antagonist. This was recently reported to significantly reduce the expected increase in kidney volume during progression of the autosomal dominantly inherited (AD) PKD (Torres et al N Engl J Med 2012 367: 2407-2418). It has only been trialled in adults, however, and more than a quarter of the patients withdrew because of side effects. This is a problem when considering human autosomal recessive (AR) PKD which affects early life /childhood where the increased water throughput may be impractical, and potentially dangerous with intercurrent childhood diseases that reduce fluid intake.

Another therapeutic approach is Sirolimus (rapamycin), an mTOR inhibitor. The effects have mainly been attributed to the antiproliferative effects of the drug (Peces et al. NDT plus 2009 2: 133-135; Tao et al J Am Soc Nephrol. 2005 16: 46-51).

Thus it can be seen that more efficacious strategies for treating cystic kidney diseases such as PKDs, particularly such as ARPKD and early ADPKD, would provide a contribution to the art.

### DISCLOSURE OF THE INVENTION

Described herein are novel treatments for cystic kidney diseases.

As described in more detail hereinafter, the present inventors have shown that large changes in the blood and lymphatic vessels occur in PKD. They have demonstrated that the microvasculature surrounding kidney cysts shifts from a blood to a lymphatic endothelial phenotype in PKD. Furthermore treatment of PKD mouse models ((Pkd1^{nl/nl} and Cys1^{cpk/cpk} mice) with a potent regulator of lymphatics (VEGF-C) significantly reduced cyst formation and enhanced growth, survival and migration of lymphatics. Thus the use of such regulators offers a therapeutic strategy for treating such diseases.

Vascular endothelial growth factor (VEGF) is a key molecule that orchestrates the formation and function of vascular networks. Impaired regulation of angiogenesis is implicated in a number of pathologic states. For instance, neoplasias exhibit uncontrolled angiogenesis, whereas ischemia and states of vascular insufficiency involve reduced VEGF activity. As the role of VEGF has been elucidated in these disease processes, its therapeutic role has been developed. The Food and Drug Administration has approved several anti-VEGF agents for treating colorectal, lung, and kidney cancer. VEGF-inducing agents have also been used experimentally to induce angiogenesis in patients with critical limb ischemia (see Birk et al Vascular and endovascular surgery 2009 42: 517-530)

A number of members of the VEGF family are currently known (VEGF A, B, C, D and E). Research into anti-VEGF agents for treating cancer through inhibition of angiogenesis has focussed predominantly on VEGF-A. Unlike VEGF-A, the factors VEGF-C and VEGF-D act predominantly on lymphangiogenesis and the development of lymphatic vasculature. VEGF-C and VEGF-D induce lymphangiogenesis via VEGFR-3 and have also been shown to be lymphangiogenic in tumours, stimulating metastasis—see Lohela et al Curr Opin Cell Biol 2009 21: 154-165.”

The present results are surprising because in the prior art it has been suggested that VEGF receptor inhibition may block cyst growth associated with cADPKD liver cyst disease—see Amura et al Am J Physiol Cell Physiol 2007 293: C419-C428. Similar suggestions have been made concerning cystic kidneys—see Tao et al Kidney Int 2007 72: 1358-1366.

Furthermore the use of mTOR inhibitors (supra) would also be expected to impede lymphangiogenesis (i.e. the opposite effect of providing a lymphangiogenic agonist)—see Huber et al Kidney Int 2007 71: 771-777.

Huang et al “Angiogenesis and autosomal dominant polycystic kidney disease.” Pediatr Nephrol. 2012 Sep. 19. [Epub ahead of print] also hypothesise that targeting pathways providing “general support” for cyst growth, such as surrounding blood vessels, may be used to reduce cyst progression. One method of achieving was postulated as VEGF-A inhibition.

VEGF-C and -D have been discussed in relation to kidney dysfunction (e.g. chronic injury and inflammation and fibrosis) and human renal biopsy specimens but not in relation to cystic kidney disease (see Lee et al Kidney Int 2012 Aug 29. doi: 10.1038/ki.2012.312. [Epub ahead of print]; Suzuki et al Kidney Int 2012 81: 865-879; Sakamoto et al. “Kidney Int 2009 75: 828-838)

Thus based on the existing art, it could not have been expected that treatment with VEGF-C or other lymphangiogenic agents could have the beneficial effects described herein in renal cystic diseases such as PKD.

Thus in one aspect of the invention there is provided a method of treating a renal cystic disease in a subject suffering therefrom, the method comprising administering a compound to the subject, wherein the compound is a lymphangiogenic agent, or a nucleic acid encoding said agent.

Lymphangiogenesis refers to formation of lymphatic vessels, particularly from pre-existing lymphatic vessels, but as used herein, the term applies to formation of lymph vessels under any condition. It also applies to the enlargement of lymphatic vessels, commonly known as lymphatic hyperplasia. Lymphangiogenesis plays an important physiological role in homeostasis, metabolism and immunity. Lymphatic vessel formation has also been implicated in a number of pathological conditions including neoplasm metastasis, oedema, rheumatoid arthritis, psoriasis and impaired wound healing.

The pro-lymphangiogenic agent, or lymphatic agonist, may be any known in the art or described herein.

Lymphangiogenesis is regulated to a large extent by VEGF-C and VEGF-D. Lymphangiogenesis appears to be regulated by signalling mediated by VEGFR-3, particularly upon specifically binding its ligands, VEGF-C and VEGF-D.

Preferably the agent is an agonist of VEGFR-3 i.e. stimulates signal transduction therefrom.

In one embodiment the agent is a VEGF-C polypeptide e.g. VEGF-C or an analog or derivative thereof.

In one embodiment the agent is a VEGF-D polypeptide e.g. VEGF-D or an analog or derivative thereof.

In one embodiment the compound is a nucleic acid encoding one of these.

In one aspect of the invention there is provided a method of treating a renal cystic disease in a subject suffering therefrom, the method comprising administering a compound to the subject, wherein the compound is an agent selected from VEGF-C or VEGF-D or an analog or derivative of either, or a nucleic acid encoding said agent.

The subject is preferably a human.

The invention also provides a compound as described for use in a method of treatment of a renal cystic disease.

The invention also provides a pharmaceutical composition comprising a compound as described, and a pharmaceutically acceptable carrier or diluent, for use in a method of treatment of a renal cystic disease.

The invention also provides use of a compound as described in the manufacture of a medicament for use in the treatment or prophylaxis of renal cystic disease.

In each case (compound, pharmaceutical or use) the disclosure herein relating to the methods of treatment will be understood to apply mutatis mutandis to these aspects also.

In the practice of aspects of the present invention, the agent will be a “selective” pro-lymphangiogenic agent, or lymphatic agonist, in the sense of acting preferentially on VEGFR-3 receptors, rather than VEGFR-1 or VEGFR-2.

As explained below, the agent may be a derivative of VEGF-C or -D for example VEGF-C¹⁵⁶ which has been engineered to act more specifically on lymphatics. In that derivative, Cys156 is replaced by a Ser residue to make it a selective agonist of VEGFR-3

The compound may be a nucleic acid which encodes an agent as described above. Such may have utility for gene therapy of renal cystic disease. This is described in more detail hereinafter.

In one embodiment the renal cystic is disease is PKD or cystic dysplasia.

In one embodiment the disease is ARPKD or ADPKD. For example the methods may be used for the early treatment of ADPKD in children.

In one embodiment the agent reduces cyst formation or number of cysts e.g. in the cortex and medulla.

In one embodiment the agent reduces the size of the cysts in the disease.

In one embodiment the agent reduces the severity of the disease, as assessed by gross kidney morphology.

In one embodiment the agent is for preserving normal renal tubules.

In one embodiment the agent is for normalising the capillary pattern or microvasculature e.g. between cortical and medullary tubules.

In one embodiment the agent is for enhancing the presence of CD31⁺ endothelia and\or VEGFR3⁺ endothelia or for inhibiting development of cyst epithelia.

The compounds described herein are believed to target the lymphatic system, and may serve to inhibit the progression of the disease. Treatment and prophylaxis is discussed in more detail below. The method may have the purpose of preventing or reducing the likelihood or severity of kidney failure or loss of renal function. The method may have the purpose of reducing kidney size/body weight ratio in the subject or cyst area. All of these outcomes can be assessed by those skilled in the art. For example kidney function may be assessed by such markers as blood urea nitrogen, serum creatinine and urinalysis.

The compounds or agents described herein may be provided in pure or isolated form for use in the methods and aspects of the invention described herein.

### Example Agents

Lymphangiogenic agents are known in the art, and can be provided and used in the light of the present specification by those skilled in the art. Similarly nucleic acids encoding said agents can be provided without undue burden.

A non-limiting list of examples of known lymphangiogenic agents includes angiopoietin-2 (http://www.uniprot.org/uniprot/015123; Gale et al. Dev Cell 2002 3: 411-423); coup-tfII (http://www.uniprot.org/uniprot/P24468, Lin et al. J Clin Invest 2010 120: 1694-1707); foxc2 (http://www.uniprot.org/uniprot/Q99958; Wu et al. Lymphology 2011 44: 35-41); neuropilin-2 (http://www.uniprot.org/uniprot/060462; Xu et al J Cell Biol 2010 188: 115-130) and prox1 (http://www.uniprot.org/uniprot/Q92786; Wigle et al EMBO J2002 21: 1505-1513);

Preferred agents are Vascular Endothelial Growth Factors (VEGFs) or analogs or derivatives. As explained above, VEGF is a sub-family of growth factors, specifically the platelet-derived growth factor family of cystine-knot growth factors. They are important signalling proteins involved in both vasculogenesis (the de novo formation of the embryonic circulatory system) and angiogenesis (the growth of blood vessels from pre-existing vasculature). Members of the platelet-derived growth factor family include the Placenta growth factor (PIGF), VEGF-A (also known as VEGF), VEGF-B, VEGF-C, VEGF-D and VEGF-E.

VEGF-A, VEGF-C and VEGF-D exert their effects by variously binding to and activating structurally related membrane receptor tyrosine kinases; VEGF receptor-1 (VEGFR-1 or Flt-I), VEGFR-2 (flk-1 or KDR), and VEGFR-3 (Flt-4). Members of the VEGF family may also interact with the structurally distinct receptor neuropilin-1 and -2. Binding of a VEGF to these receptors initiates a signaling cascade, resulting in effects on gene expression and cell survival, proliferation, and migration.

It is known in the art that Vascular endothelial growth factor-C and -D drive lymphangiogenesis through VEGFR-3 and partly through VEGFR-2.

VEGF-D is reported to be a more potent lymphatic agonist than VEGF-C (Rissanen et al. Circ Res 2003 92: 1098-1106).

Preferred agents are therefore human VEGF-C or -D. These agents are well characterised and their sequences are known in the art (and set out herein as SEQ ID No.s 1 and 2 respectively).

VEGF-C has been proposed for therapeutic lymphangiogenesis, albeit not in cystic kidney disease—see Szuba et al. “Therapeutic lymphangiogenesis with human recombinant VEGF-C.” The FASEB journal 2002 16: 1985-1987; Goldman et al. “Regulation of lymphatic capillary regeneration by interstitial flow in skin.” American Journal of Physiology-Heart and Circulatory Physiology 2007: 292: H2176-H2183.

Other lymphangiogenic agents are those which bind to and stimulate or induce signaling mediated by VEGFR-3. These will preferably bind selectively to that receptor. By “selectively binds VEGFR-3” is meant that the polypeptide fails to significantly bind VEGFR -2 and is not proteolytically processed in vivo into a form that shows significant reactivity with VEGFR-2. An exemplary VEGFR-3 specific VEGF-C polypeptide comprises a VEGF-C 156 polypeptide described below.

The term “VEGF-C polypeptide” includes any polypeptide that has a VEGF-C or VEGF-C analog amino acid sequence (i.e. a variant amino acid sequence, as defined elsewhere herein in greater detail) and that possesses VEGFR-3 binding and stimulatory properties (i.e. causes lymphangiogenesis). The term “VEGF-C polynucleotide” includes any polynucleotide (e.g., DNA or RNA, single- or double-stranded) comprising a nucleotide sequence that encodes a VEGF-C polypeptide. Due to the well-known degeneracy of the genetic code, multiple VEGF-C polynucleotide sequences encode any selected VEGF-C polypeptide. Derivatives which may be useful in the present invention are described, for example, in WO9705250 the contents of which are explicitly incorporated herein, and references cited therein.

In one embodiment the agent is a derivative which has been modified to enhance activity, specificity, or any other pharmacokinetic property e.g. half-life.

A preferred derivative is VEGF-C¹⁵⁶ where Cys156 is replaced by a Ser residue (or another residue) which reportedly increases it selectivity for VEGFR-3 (Joukov et al J Biol Chem 1998 273: 6599-6602.

### Variants of Polypeptides and Nucleic Acids

It will be understood by those skilled in the art that functional variants derived from the sequences discussed above may likewise be employed in the present invention.

Preferred functional derivatives of the agent include proteins that may comprise mutations (relative to the wild type) that nevertheless do not alter the activity of the agent. In accordance with the present invention, preferred further changes in the agent are commonly known as “conservative” or “safe” substitutions. Conservative amino acid substitutions are those with amino acids having sufficiently similar chemical properties, in order to preserve the structure and the biological function of the agent. It is clear that insertions and deletions of amino acids may also be made in the above defined sequences without altering their function, particularly if the insertions or deletions only involve a few amino acids, e.g. under ten and preferably under five, and do not remove or displace amino acids which are critical to the functional confirmation of the agent. The literature provide many models on which the selection of conservative amino acids substitutions can be performed on the basis of statistical and physico-chemical studies on the sequence and/or the structure of a natural protein.

It will be appreciated by the skilled technician that functional derivatives of the amino acid, and nucleic acid sequences, disclosed herein, may have a sequence which has at least 30%, preferably 40%, more preferably 50%, and even more preferably, 60% sequence identity with the amino acid/polypeptide/nucleic acid sequences of any of the sequences referred to herein. An amino acid/polypeptide/nucleic acid sequence with a greater identity than preferably 65%, more preferably 75%, even more preferably 85%, and even more preferably 90% to any of the sequences referred to is also envisaged. Preferably, the amino acid/polypeptide/nucleic acid sequence has 92% identity, even more preferably 95% identity, even more preferably 97% identity, even more preferably 98% identity and, most preferably, 99% identity with any of the referred to sequences.

In another embodiment, the compound is or comprises a polypeptide which comprises an amino acid sequence at least 80%, at least 85%, at least 90%, at least 91%, at least 92%, at least 93%, at least 94%, at least 95%, at least 96%, at least 97%, at least 98%, and least 99% or more identical to the amino acid sequence set forth in SEQ ID NO: 1 or 2 or to a fragment thereof that binds VEGFR-3, where the polypeptide or fragment binds to VEGFR-3.

In another embodiment relevant to gene therapy, the compound comprises a polynucleotide that encodes a polypeptide comprising an amino acid sequence at least 80%, at least 85%, at least 90%, at least 91%, at least 92%, at least 93%, at least 94%, at least 95%, at least 96%, at least 97%, at least 98%, and least 99% or more identical to the amino acid sequence set forth in SEQ ID NO: 1 or 2 or a fragment thereof, where the polypeptide or fragment binds to VEGFR-3.

Calculation of percentage identities between different amino acid/polypeptide/nucleic acid sequences may be carried out as follows. A multiple alignment is first generated by the ClustaIX program (pair wise parameters: gap opening 10.0, gap extension 0.1, protein matrix Gonnet 250, DNA matrix IUB; multiple parameters: gap opening 10.0, gap extension 0.2, delay divergent sequences 30%, DNA transition weight 0.5, negative matrix off, protein matrix gonnet series, DNA weight IUB; Protein gap parameters, residue-specific penalties on, hydrophilic penalties on, hydrophilic residues GPSNDQERK, gap separation distance 4, end gap separation off). The percentage identity is then calculated from the multiple alignment as (N/T)*100, where N is the number of positions at which the two sequences share an identical residue, and T is the total number of positions compared. Alternatively, percentage identity can be calculated as (N/S)*100 where S is the length of the shorter sequence being compared. The amino acid/polypeptide/nucleic acid sequences may be synthesised de novo, or may be native amino acid/polypeptide/nucleic acid sequence, or a derivative thereof.

Alternatively, a substantially similar nucleotide sequence will be encoded by a sequence which hybridizes to any of the nucleic acid sequences referred to herein or their complements under stringent conditions. By stringent conditions, we mean the nucleotide hybridises to filter-bound DNA or RNA in 6× sodium chloride/sodium citrate (SSC) at approximately 45° C. followed by at least one wash in 0.2×SSC/0.1% SDS at approximately 5-65° C. Alternatively, a substantially similar polypeptide may differ by at least 1, but less than 5, 10, 20, 50 or 100 amino acids from the peptide sequences according to the present invention.

Due to the degeneracy of the genetic code, it is clear that any nucleic acid sequence could be varied or changed without substantially affecting the sequence of the agent protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid.

Exemplary VEGF-C and VEGF-D variants suitable for use in the present invention are described in WO2008096268.

In addition to sequence variants, various modifications to protein sequences are also envisaged and within the scope of the claimed invention, i.e. those which occur during or after translation, e.g. by acetylation, amidation, carboxylation, phosphorylation, proteolytic cleavage or linkage to a ligand.

Derivatives of protein or peptide agents used according to the invention include derivatives that increase the half-life of the agent in vivo. Examples of derivatives capable of increasing the half-life of polypeptides according to the invention include peptoid derivatives, D-amino acid derivatives and peptide-peptoid hybrids.

Proteins and peptide agents according to the present invention may be subject to degradation by a number of means (such as protease activity at a target site). Such degradation may limit their bioavailability and hence therapeutic utility. There are a number of well-established techniques by which peptide derivatives that have enhanced stability in biological contexts can be designed and produced. Such peptide derivatives may have improved bioavailability as a result of increased resistance to protease-mediated degradation. Preferably, a derivative suitable for use according to the invention is more protease-resistant than the protein or peptide from which it is derived. Protease-resistance of a peptide derivative and the protein or peptide from which it is derived may be evaluated by means of well-known protein degradation assays. The relative values of protease resistance for the peptide derivative and peptide may then be compared.

Peptoid derivatives of proteins and peptides according to the invention may be readily designed from knowledge of the sequences described herein or known in the art. Commercially available software may be used to develop peptoid derivatives according to well-established protocols.

Retropeptoids, (in which all amino acids are replaced by peptoid residues in reversed order) are also able to mimic proteins or peptides according to the invention. A retropeptoid is expected to bind in the opposite direction in the ligand-binding groove, as compared to a peptide or peptoid-peptide hybrid containing one peptoid residue. As a result, the side chains of the peptoid residues are able to point in the same direction as the side chains in the original peptide.

A further embodiment of a modified form of peptides or proteins according to the invention comprises D-amino acid forms. In this case, the order of the amino acid residues is reversed. The preparation of peptides using D-amino acids rather than L-amino acids greatly decreases any unwanted breakdown of such derivative by normal metabolic processes, decreasing the amounts of the derivative which needs to be administered, along with the frequency of its administration.

### Nucleic Acids\Gene Therapy

Instead of administering the agents described herein directly, the agents may be produced in the target cells by expression from a heterologous encoding gene introduced into the cells, e.g. in a suitable vector. The vector could be targeted to the specific cells to be treated, or it could contain regulatory elements which are switched on more or less selectively by the target cells. Thus nucleic acid-based therapeutics of the invention may be used in place of polypeptides or oligomers as “naked DNA” or in with conventional gene therapy vectors, such as are well known in the art.

The term “heterologous” is used broadly in this aspect to indicate that the gene/sequence of nucleotides in question (e.g. encoding a VEGF-C polypeptide) have been introduced into said cells of the kidney or cyst artificially i.e. by human intervention. A heterologous gene may be identical to an endogenous equivalent gene.

Therefore in one aspect of the present invention, the nucleic acid encoding the agent for use in the method is in the form of a recombinant and preferably replicable vector.

As explained below, expression vectors derived from retroviruses, adenoviruses, or herpes or vaccinia viruses, or from various bacterial plasmids, may be used for delivery of nucleotide sequences to the targeted organ, tissue, or cell population. Methods which are well known to those skilled in the art can be used to construct vectors to express the DNA molecules of the invention.

Generally speaking, those skilled in the art are well able to construct vectors and design protocols for recombinant gene expression. Suitable vectors can be chosen or constructed, containing, in addition to the elements of the invention described above, appropriate regulatory sequences, including promoter sequences, terminator fragments, polyadenylation sequences, marker genes and other sequences as appropriate. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, (1995, and periodic supplements).

In mammalian cells, a number of viral-based expression systems may be utilized e.g. adenovirus, SV40 or EBV-based vectors are all well known to those skilled in the art. Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes. By way of further example, constructs of the present invention capable of increasing expression of the target protein can be administered to the subject either as a naked polynucleotide or formulated with a carrier, such as a liposome, to facilitate incorporation into a cell. Such constructs can also be incorporated into appropriate vaccines, such as in viral vectors (e.g. vaccinia), bacterial constructs, such as variants of the well-known BCG vaccine, and so forth.

VEGFR3 has previously been targeted for gene therapy in the art, albeit not for the treatment of cystic kidney diseases -see eg. Szuba et al., “Therapeutic lymphangiogenesis with human recombinant VEGF-C,” FASEB J. 2002 16: 1985-1987; and Yoon et al., “VEGF-C gene therapy augments postnatal lymphangiogenesis and ameliorates secondary lymphedema,” J. Clin. Invest. 2003 111: 717-725.

VEGF-C gene therapy is described in WO2008/096268 of Vegenics Ltd, the entire disclosure of which is specifically incorporated herein. Examples of polynucleotides described therein include a nucleotide sequence encoding a secretory signal peptide, wherein the sequence encoding the secretory signal peptide is connected in-frame with the sequence that encodes the VEGF-C polypeptide. The polynucleotide may further comprise a promoter and/or enhancer sequence operably connected to the sequence that encodes the secretory signal sequence and VEGF-C polypeptide, wherein the promoter sequence promotes transcription of the sequence that encodes the secretory signal sequence and the VEGF-C polypeptide in cells of the mammalian subject. In one variation, the promoter is a constitutive promoter that promotes expression in a variety of cell types, such as the cytomegalovirus promoter/enhancer (Lehner et al, J. Clin. Microbiol., 29:2494-2502 (1991); Boshart et al, Cell, 41:521-530 (1985)); or Rous sarcoma virus promoter (Davis et al, Hum. Gene Ther., 4:151 (1993)) or simian virus 40 promoter. Also contemplated is an endothelial cell specific promoter such as Tie promoter (Korhonen et al, Blood, 86(5): 1828-1835 (1995); U.S. Pat. No. 5,877,020).

By “promoter” is meant a sequence of nucleotides from which transcription may be initiated of DNA operably linked downstream (i.e. in the 3′ direction on the sense strand of double-stranded DNA).

“Operably linked” means joined as part of the same nucleic acid molecule, suitably positioned and oriented for transcription to be initiated from the promoter. DNA operably linked to a promoter is “under transcriptional initiation regulation” of the promoter.

As noted in WO2008/096268, a variety of vectors are suitable to introduce the VEGF-C (or VEGF-D) transgene into the host. Exemplary vectors that have been described in the literature include replication-deficient retroviral vectors, including but not limited to lentivirus vectors (Kim et al, J. Virol, 72(1): 811-816 (1998); Kingsman & Johnson, Scrip Magazine, October, 1998, pp. 43-46.); adeno-associated viral vectors (Gnatenko et al, J. Investig. Med., 45: 87-98 (1997)); adenoviral vectors (See, e.g., U.S. Pat. No. 5,792,453; Quantin et al, Proc. Natl. Acad. Sci. USA, 89: 2581-2584 (1992); Stratford-Perricadet et al, J. Clin. Invest., 90: 626-630 (1992); and Rosenfeld et al, Cell, 68: 143-155 (1992)); Lipofectin-mediated gene transfer (BRL); liposomal vectors (See, e.g., U.S. Pat. No. 5,631,237 (Liposomes comprising Sendai virus proteins)); and combinations thereof. Additionally, the VEGF-C (or VEGF-D) transgene can be transferred via particle-mediated gene transfer (Gurunluonglu, R., et al, Ann. Plast. Surg., 49:161-169 (2002)). Additional or alternative example gene therapy vectors for use in the method of this invention include retroviral or episomal vectors expressing particular desired genes under the control of the promoter and/or the supplemental control sequences (see, e.g., Axel, et al., U.S. Pat. No. 4,399,216, and Pastan, et al., U.S. Pat. No. 5,166,059; also WO0159142 all incorporated herein by reference). Delivery systems as contemplated herein include both viral and liposomal delivery systems (see, e.g., Davis, et al., U.S. Pat. No. 4,920,209, incorporated herein by reference). All of the foregoing documents are incorporated herein by reference in the entirety.

Thus one DNA based therapeutic approach provided by the present invention is the use of a vector which comprises one or more nucleotide sequences encoding one of the agents described herein.

### Formulations, Route of Administration and Dosage Regimes

As described above, the present invention pertains to a method of treatment of cystic kidney disease in the subject, the method comprising administering to said subject a prophylactically or therapeutically effective amount of a compound as described herein, preferably in the form of a pharmaceutical composition.

The compounds in the present invention may be given prophylactically in respect of cyst reduction or treatment, which may otherwise follow onset of the disease.

The term “treatment,” as used herein in the context of treating a condition, pertains generally to treatment and therapy of a human, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount,” as used herein, pertains to that amount of a compound of the invention, or a material, composition or dosage from comprising said compound, which is effective for producing some desired therapeutic effect, commensurate with a reasonable benefit/risk ratio, when administered in accordance with a desired treatment regimen.

Similarly, the term “prophylactically effective amount,” as used herein pertains to that amount of a compound of the invention, or a material, composition or dosage from comprising said compound, which is effective for producing some desired prophylactic effect, commensurate with a reasonable benefit/risk ratio, when administered in accordance with a desired treatment regimen.

“Prophylaxis” in the context of the present specification should not be understood to circumscribe complete success i.e. complete protection or complete prevention. Rather prophylaxis in the present context refers to a measure which is administered in advance of detection of a symptomatic condition with the aim of preserving health by helping to delay, mitigate or avoid that particular condition.

While it is possible for the compound of the invention to be used (e.g., administered) alone, it is often preferable to present it as a composition or formulation.

Another aspect of the invention therefore provides a composition comprising a compound as described herein, and a pharmaceutically acceptable carrier or diluent.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

In some embodiments, the composition is a pharmaceutical composition (e.g., formulation, preparation, medicament) comprising, or consisting essentially of, or consisting of as a sole active ingredient, a compound as described herein, and a pharmaceutically acceptable carrier, diluent, or excipient.

In some embodiments, the composition is a pharmaceutical composition comprising at least one compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

In some embodiments, the composition further comprises other active agents, for example, other therapeutic or prophylactic agents. This is discussed in more detail below.

In accordance with the present invention, a preferred route of administration is injection direct into the target site.

Formulations suitable for parenteral administration (e.g., by injection), include aqueous or non-aqueous, isotonic, pyrogen-free, sterile liquids (e.g., solutions, suspensions), in which the compound is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the compound in the liquid is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

Other dosage forms include a powder, tablet, capsule, liquid, ointment, cream, gel, hydrogel, aerosol, spray, micelle, transdermal patch, liposome or any other suitable form that may be administered to a person or animal. It will be appreciated that the vehicle of the composition of the invention should be one which is well tolerated by the subject to whom it is given and enables delivery of the compounds to the site of action i.e. kidney. Other carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994.

Transdermal patches suitable for the administration of a compound or composition of the invention are described in WO2008096268, wherein the patch comprises a composition comprising a VEGF-C polynucleotide, a VEGF-C polypeptide, a VEGF-D polynucleotide, and/or a VEGF-D polypeptide. The thickness of the transdermal patch depends on the therapeutic requirements and may be adapted accordingly.

The formulations may be prepared by any methods well known in the art of pharmacy. Such methods include the step of bringing into association the compound with a carrier which constitutes one or more accessory ingredients. In general, the formulations are prepared by uniformly and intimately bringing into association the compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician or clinician.

For example the treatments described herein may be given by weekly injection.

Based on the results in the Examples suitable doses may optionally be in the range of about 5 μg/kg to about 10 mg/kg of the subject, more preferably about 50 μg/kg to about 1 mg/kg, for example about 100 μg/kg, to lead to a therapeutic response in patients. Similar doses of other growth factors (i.e.) insulin-like growth factor have been utilised in humans, see for example Goeters et al. Ann Surg 1995 222: 646-653; Cheetham et al. Diabet Med 1995 12: 885-892.

As described in WO2008096268, in gene therapy embodiments employing viral delivery, the unit dose may be calculated in terms of the dose of viral particles being administered.

Viral doses include a particular number of virus particles or plaque forming units (pfu). For embodiments involving adenovirus, particular unit doses include 103, 104, 105, 106, 107, 108, 109, 1010, 1011, 1012, 1013 or 1014 pfu. Particle doses may be somewhat higher (10 to 100 fold) due to the presence of infection-defective particles.

### Combination Therapies

In some embodiments the methods or treatments of the present invention may be combined with other therapies, whether symptomatic or disease modifying.

Combining endothelial therapies, such as those described herein, with therapies targeted at cyst epithelia may be particularly desirable.

The term “treatment” includes combination treatments and therapies, in which two or more treatments or therapies are combined, for example, sequentially or simultaneously.

For example it may be beneficial to combine treatment with a compound as described herein with one or more other (e.g., 1, 2, 3, 4) agents or therapies.

Appropriate examples of co-therapeutics will be known to those skilled in the art on the basis of the disclosure herein. Typically the co-therapeutic may be any known in the art which it is believed may give therapeutic effect in treating the diseases described herein.

Other co-therapeutics may be alternate agents which stimulate lymphangiogenesis.

A non-limiting list of co-therapeutics growth factors or other lymphatic stimulators such as angiopoietin-2 (http://www.uniprot.org/uniprot/015123; Gale et al. Dev Cell 2002 3: 411-423); coup-tfII (http://www.uniprot.org/uniprot/P24468, Lin et al. J Clin Invest 2010 120: 1694-1707); foxc2 (http://www.uniprot.org/uniprot/Q99958; Wu et al. Lymphology 2011 44: 35-41); neuropilin-2 (http://www.uniprot.org/uniprot/O60462; Xu et al J Cell Biol 2010 188: 115-130) and prox1 (http://www.uniprot.org/uniprot/Q92786; Wigle et al EMBO J 2002 21: 1505-1513); drugs which target cyst proliferation such as rapamycin, vasopressin antagonists (Tao et al J Am Soc Nephrol. 2005 16: 46-5; Torres et al N Engl J Med 2012 367: 2407-2418) or drugs that treat progression of kidney disease such as ACE inhibitors and Angiotensin II antagonists (Jafar et al Kidney Int 2005 67: 265-271; Torres et al Kidney Int 2012 81: 577-585).

The particular combination would be at the discretion of the physician who would also select dosages using his/her common general knowledge and dosing regimens known to a skilled practitioner.

The agents (i.e., a compound as described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

The agents (i.e., a compound as described here, plus one or more other agents) may be formulated together in a single dosage form, or alternatively, the individual agents may be formulated separately and presented together in the form of a kit, optionally with instructions for their use.

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

### FIGURES

FIG. 1. Administration of VEGFC to Cys1^{cpk/cpk} mice

(a) Outline of experimental strategy. (b) Representative images showing overall appearance of kidneys from Cys1^{+/+} and Cys1^{cpk/cpk} mice administered either PBS or VEGFC. Bar is 0.5 cm for each panel. (c) Assessment of kidney/body weight ratio and blood urea nitrogen concentration (d). Representative images of periodic-acid Schiff stained kidney sections obtained from Cys1^{+/+} and Cys1^{cpk/cpk} mice administered either PBS or VEGFC. Bar is 50 μm in each panel (e-j). Results of Image J particle analysis of images of whole kidneys under a dissection microscope to determine the average area of individual cysts (k) n=7-11 in each group and analyses, *=p<0.05, **=p<0.01, ***=p<0.001 between groups.

FIG. 2. Administration of VEGFC to Pkd1^{nl/nl} mice

(a) Outline of experimental strategy. (b) Representative images showing overall appearance of kidneys from Pkd1^{+/+} and Pkd1^{nl/nl} mice administered either PBS or VEGFC. Bar is 0.5 cm for each panel. (c) Assessment of kidney/body weight ratio and blood urea nitrogen concentration (d). Representative images of periodic-acid Schiff stained kidney sections obtained from Pkd1^{+/+} and Pkd1^{nl/nl} mice administered either PBS or VEGFC. Bar is 50 μm in each panel (e-j). Results of Image J particle analysis of images of whole kidneys under a dissection microscope to determine the average area of individual cysts (k). n=4-8 in each group and analyses, *=p<0.05, between groups.

FIG. 3. Effects of VEGFC administration on the renal microvasculature in Pkd1^{nl/nl} mice

Pkd1^{+/+} mice contained CD31⁺ and VEGFR3⁺ vessels arranged in a fine linear network surrounding cortical and medullary tubules (a-d); these patterns were disrupted in untreated Pkd1^{nl/nl} (e-h) whilst VEGFC administration to Pkd1^{nl/nl} mice normalised these aberrant patterns (i-l). Images are representative of 5-8 animals analysed in each group. Quantification of proliferating VEGFR3 (m) and CD31 (n) capillaries and CD206 (o) positive cells, n=5-8 in each group, *=p<0.05 between groups. Neither VEGFR3 (p) or VEGFR2 (q) were expressed in the wall of cysts (cy) originating from the distal tubules which were positive for galectin-3. (r-t) Schematic diagram outlining the changes in the renal microvasculature in PKD kidneys and the effect of VEGFC therapy. Bar is 50 μm in each panel.

```
Sequence of VEGF-C: SEQ ID 1
http://www.uniprot.org/uniprot/P49767
                10 20 30 40
MHLLGFFSVA CSLLAAALLP GPREAPAAAA AFESGLDLSD
                50 60 70 80
AEPDAGEATA YASKDLEEQL RSVSSVDELM TVLYPEYWKM
                90 100 110 120
YKCQLRKGGW QHNREQANLN SRTEETIKFA AAHYNTEILK
              130 140 150 160
SIDNEWRKTQ CMPREVCIDV GKEFGVATNT FFKPPCVSVY
              170 180 190 200
RCGGCCNSEG LQCMNTSTSY LSKTLFEITV PLSQGPKPVT
              210 220 230 240
ISFANHTSCR CMSKLDVYRQ VHSIIRRSLP ATLPQCQAAN
              250 260 270 280
KTCPTNYMWN NHICPCLAQE DFMFSSDAGD DSTDGFHDIC
              290 300 310 320
GPNKELDEET CQCVCRAGLR PASCGPHKEL DRNSCQCVCK
              330 340 350 360
NKLFPSQCGA NREFDENTCQ CVCKRTCPRN QPLNPGKCAC
              370 380 390 400
ECTESPQKCL LKGKKFHHQT CSCYRRPCTN RQKACEPGFS
              410
YSEEVCRCVP SYWKRPQMS
Sequence of VEGF-D: SEQ ID 2
http://www.uniprot.org/uniprot/043915
                10 20 30 40
MYREWVVVNV FMMLYVQLVQ GSSNEHGPVK RSSQSTLERS
                50 60 70 80
EQQIRAASSL EELLRITHSE DWKLWRCRLR LKSFTSMDSR
                90 100 110 120
SASHRSTRFA ATFYDIETLK VIDEEWQRTQ CSPRETCVEV
              130 140 150 160
ASELGKSTNT FFKPPCVNVF RCGGCCNEES LICMNTSTSY
              170 180 190 200
ISKQLFEISV PLTSVPELVP VKVANHTGCK CLPTAPRHPY
              210 220 230 240
SIIRRSIQIP EEDRCSHSKK LCPIDMLWDS NKCKCVLQEE
              250 260 270 280
NPLAGTEDHS HLQEPALCGP HMMFDEDRCE CVCKTPCPKD
              290 300 310 320
LIQHPKNCSC FECKESLETC CQKHKLFHPD TCSCEDRCPF
              330 340 350
HTRPCASGKT ACAKHCRFPK EKRAAQGPHS RKNP
```

### EXAMPLES

### Materials

Experiments were performed in the congenital polycystic kidney (cpk) mouse and pkd1-hypomorphic (pkd1hm) mouse, models of ARPKD and ADPKD respectively (Chiu et al. Am J Pathol 2006 169: 1925-1938; Lantinga van-Leeuwen et al Hum Mol Genet 2004 13: 3069-3077).

### Example 1

### Assessment of Renal Blood and Lymphatic Vasculature in cpk Mice

### Methods

We investigated the renal blood and lymphatic vasculature in congenital polycystic kidney (cpk) mice; a model of autosomal recessive PKD (ARPKD) by real-time PCR and immunohistochemistry through different stages of disease progression. The cpk mouse gives a phenotype which is recessively inherited and appears clinically similar to human ARPKD, although it is caused by a mutation in cystin rather than pkhdl which underlies the human disease

### Results

Surrounding the smaller cortical cysts of cpk mice, the blood vasculature was more prominent than in wild-type littermates with intense CD31 staining; structurally, these vessels were dilated and disorganised. In larger medullary cysts, there was regression of the blood vasculature. This was accompanied by reduced kidney mRNA levels of endothelial markers Vegfr1, Vegfr2, Tie1, Tie2 and Pv1. Using VEGFR-3 immunostaining, the lymphatic vasculature was more pronounced in cpk mice compared to wild-type littermates and mRNA levels of LYVE-1 and podoplanin were upregulated.

### Conclusion

The vasculature in PKD is disorganised with changes in the balance between blood and lymphatic vessels.

### Example 2

### Treatment of PKD Mouse Models with Factor Modifying Lymphanqioqenesis

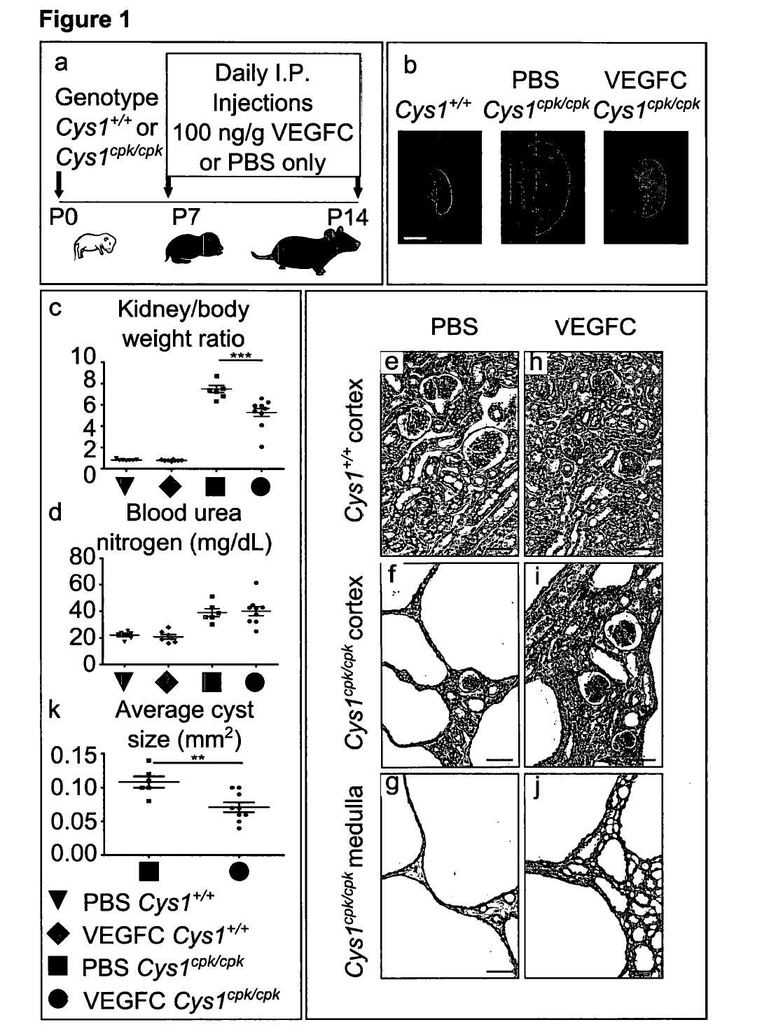

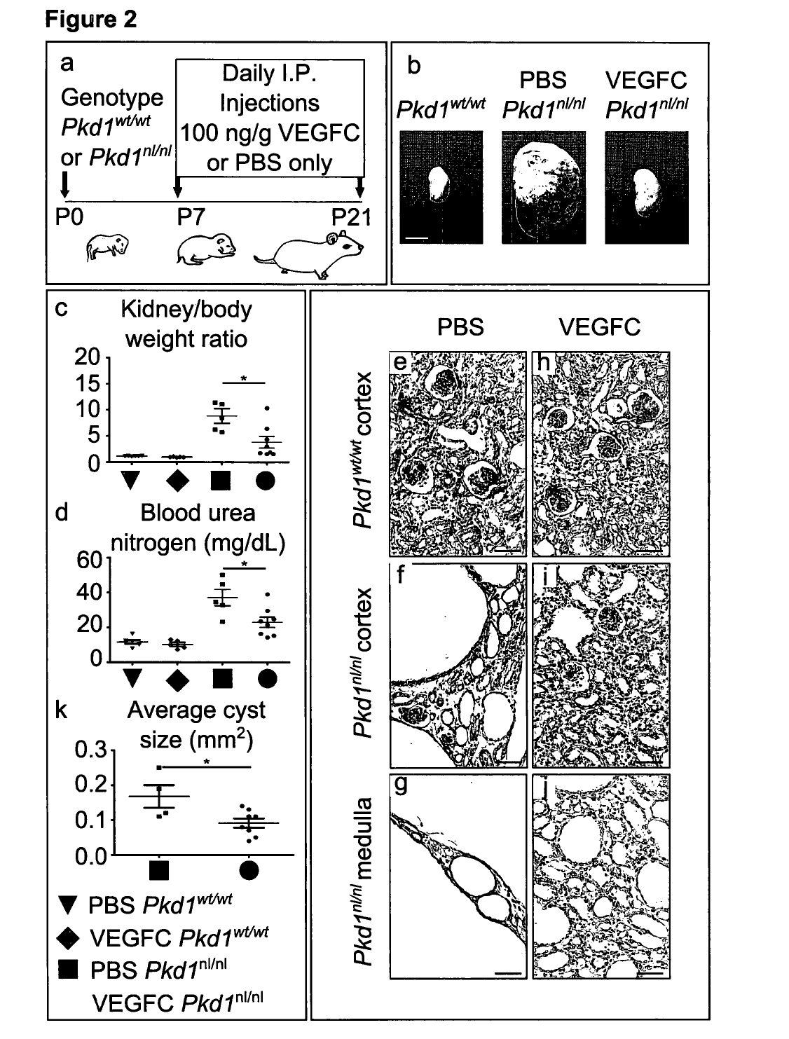

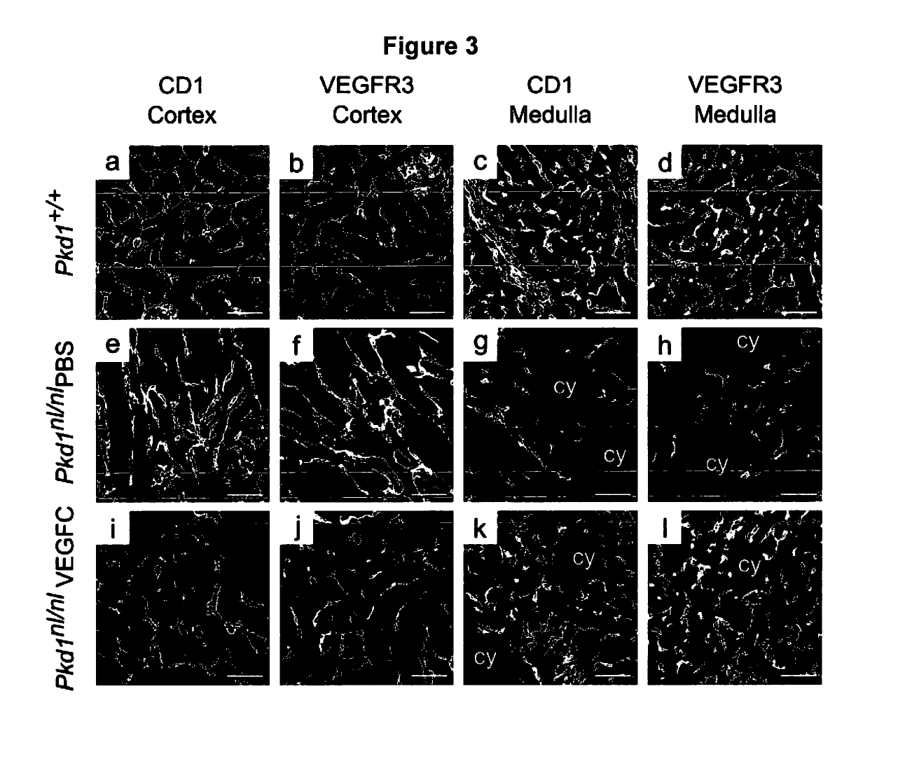

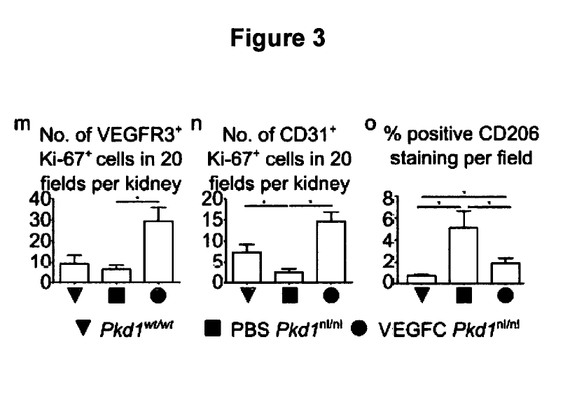

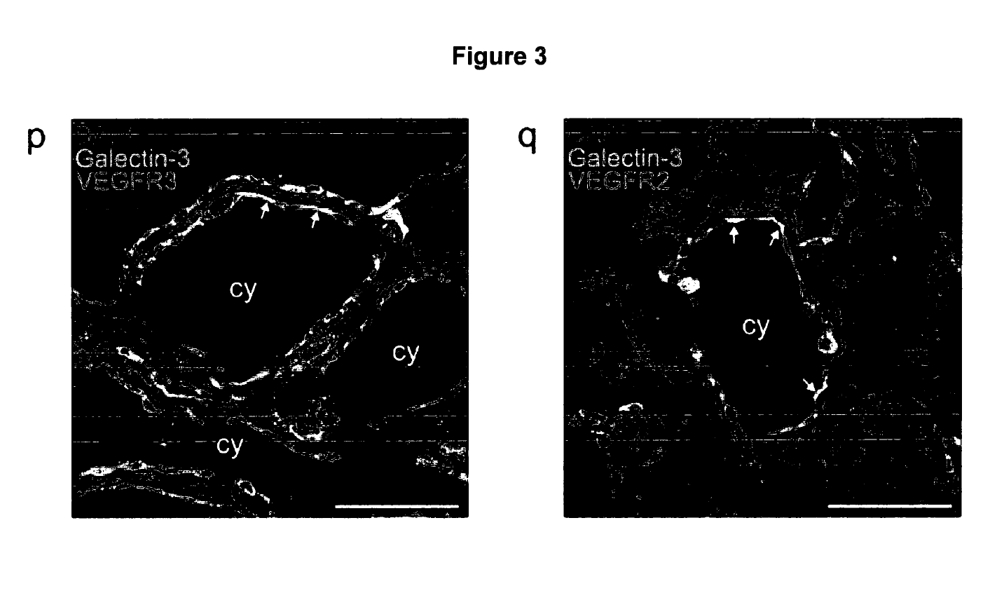

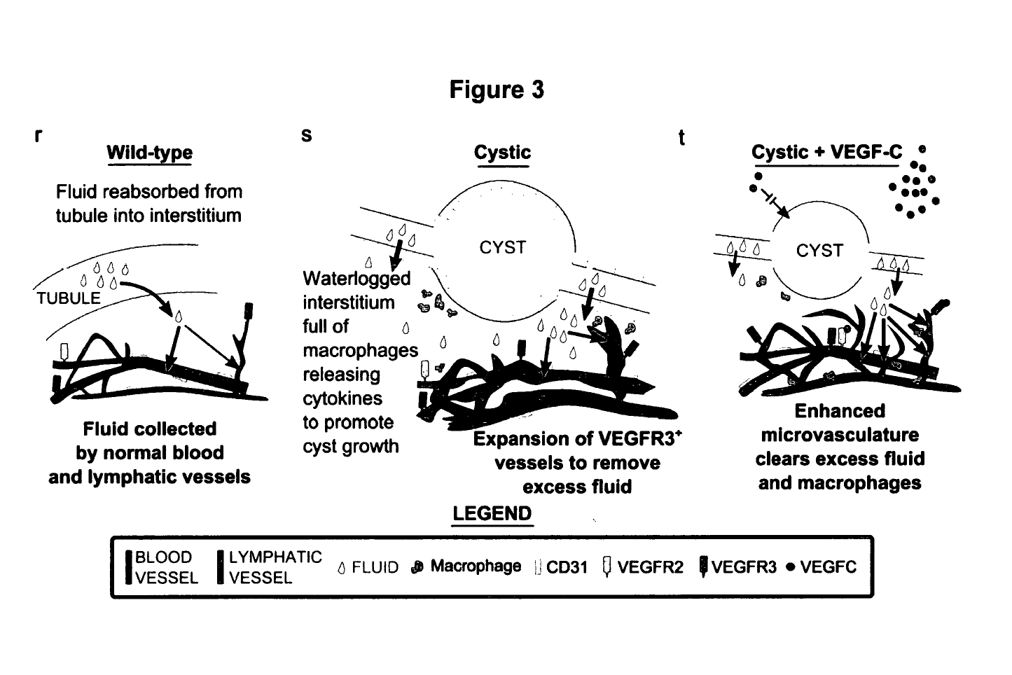

We administered 100 ng/g body weight of recombinant VEGFC intraperitionally to Cys1^{cpk/cpk} mice, a model of autosomal recessive (AR)PKD daily from day 7 for one week (FIG. 1a). VEGFC-treated Cys1^{cpk/cpk} mice had reduced severity of PKD as assessed by gross morphology (FIG. 1b) and a significant reduction in kidney/body weight ratio (7.5%±0.4 and 5.3±0.6 in Cys1^{cpk/cpk} administered PBS and VEGFC, p<0.001, FIG. 1c). VEGFC treatment did not, however, affect blood urea nitrogen concentration, a measure of renal excretory function (FIG. 1d). On histology, VEGFC-treated animals had less prominent cysts in the cortex and medulla (FIG. 1f-g,i-j) and led to a significantly smaller average cyst size (0.11 mm²±0.01 and 0.07±0.01 in Cys1^{cpk/cpk} administered PBS and VEGFC, p<0.01, FIG. 1k). Cys1^{+/+} mice administered VEGFC showed no ill-effects of the treatment (FIG. 1c,h).

Next we performed experiments using Pkd1^{nl/nl} mice, which carry two hypomorphic alleles of Pkd1; the mouse homologue of the gene most commonly mutated in human ADPKD. We treated Pkd1^{nl/nl} with recombinant VEGFC, focusing on weeks 1-3 (FIG. 2a). VEGFC improved gross morphology (FIG. 2b) and significantly reduced kidney/body weight ratio (8.6%±1.3 and 3.8±1.6 in Pkd1^{nl/nl} administered PBS and VEGFC, p<0.05, FIG. 2c). It also significantly reduced blood urea nitrogen concentration in these Pkd1^{nl/nl} mice (37.1 mg/dL±5.3 and 23.0±4.2 in Pkd1^{nl/nl} administered PBS and VEGFC, p<0.05, FIG. 2d) and preserved their normal renal tubules (FIG. 2f-g,i-j). VEGFC also significantly decreased the average size of each cyst (0.17 mm²±0.03 and 0.09±0.02 in Pkd1^{nl/nl} administered PBS and VEGFC, p<0.05, FIG. 2k) in Pkd1^{nl/nl} mice.

CD31⁺ and VEGFR3⁺ capillaries located between cortical and medullary tubules in untreated Pkd1^{nl/nl} mice showed changes in patterns compared with wild types (FIG. 3a-h). VEGFC treatment of Pkd1^{nl/nl} mice normalised these aberrant patterns (FIG. 3i-l). This was associated with proliferation of CD31⁺ endothelia and VEGFR3⁺ endothelia (FIG. 3m,n) and a significant reduction of CD206⁺ macrophages (FIG. 3o), the latter cells being functionally implicated in PKD cyst growth. We could not detect VEGFR2 in cyst epithelia, nor was VEGFR3 immunodetected in these cells (FIG. 3p,q), arguing against a direct effect of VEGFRC on cyst growth.

### Conclusion

Treatment targeting the renal vasculature may be a novel therapy for both ARPKD and ADPKD. All treated mice survived and looked healthy, but their kidney size and average cyst size was approximately half that of their untreated peers.

### Example 3

### Use of Alternative Factors and Gene Therapy

### Materials and Methods

Experiments are performed in mouse models of ARPKD and ADPKD respectively. VEGF-D is administered initially as recombinant protein using the dosing regimen that we found to be successful for VEGF-C (see Example 2) and is compared with VEGF-C¹⁵⁶ engineered to act specifically on lymphatics (Joukov et al. 1998).

Different dose regimens and adenovirus gene therapy which we have previously used to overexpress vascular growth factors in mice (Long et al Kidney Int 2008 74: 300-309) are also utilised.

Specifically, the adenovirus systems (e.g. from Regeneron Pharmaceuticals) may be used to over-express genes of interest. These can be used to growth factors such as angiopoietins (Long et al Kidney Int 2008 74: 300-309). One injection per animal generates expression within 1-2 days that lasted for three weeks.

Experimental time course and regimen will reflect rapid cyst development in cpk and slower progression in pkd1hm animals. Primary outcome will be rate of PKD progression as assessed by renal function, kidney size and cyst area, plus toxicity assessment to ensure VEGF therapy is safe. Supporting parameters will be expression/levels of VEGF-D, -C and other VEGFs; assessment of lymphatic and blood vessel density and distribution using immunohistochemistry and in situ hybridisation; and measurement of gene expression changes covering a wide range of lymphatic/vascular and PKD-associated molecules using a rapid cost-effective targeted RT² profiler PCR array. Changes detected by the latter technique is confirmed by in situ hybridisation, immunohistochemistry and/or western blotting.

### Treatment Strategy

### (i) cpk Mice.

Groups: Daily injections from day 7 to 14 of i) VEGF-D, ii) VEGF-C¹⁵⁶ or iii) PBS

Sacrifice 6-8 animals in each group at 2 and 3 weeks; allow others to continue and kill later to assess prolonged survival in cpk. Assess further side effects over longer period of 3 and 6 months in normal mice

Therapy must be started early because cpk mice are starting to get cysts by day 7, have massive cystic proliferation between day 8-14 and die by 3-4 weeks. Direct protein injection is used as per in the experimental protocol outlined above. Neonatal cpk mice are injected with VEGF-D or VEGF-C¹⁵⁶ (active treatment groups), or phosphate buffered saline (PBS; control) intraperitoneally daily; this regimen replicates our earlier VEGF-C experiment. Additionally, in some experiments, the survival time of the cpk mice is assessed (within health assessment limits set by the UK Home Office) to determine how long lifespan is extended by these therapies. Half of the normal heterozygote cpk and wild-type littermates are left for 3 or 6 months to monitor for tumour formation and other side effects. Mice are placed in metabolic cages before sacrifice to collect urine for 24 hr analysis, and blood urea nitrogen, serum creatinine to compare effects on renal function. VEGF levels will also be measured.

(ii) pkd1hm Mice: Short-Term; Prevention of Formation.

Therapy is again be initiated on day 7, and will be given every day up to 3 weeks. The experimental protocol is outlined below, with three groups replicating the cpk experiment.

Mice will be sacrificed at the end of therapy (i.e. 3 weeks), 6 and 9 weeks, again preceded by 1 day in metabolic cages and blood samples as above.

(ii) pkd1hm Mice: Longer-Term; Treatment of Developed Cysts

Therapies begin at week 4 when a reasonable number of cysts are expected. Groups reiterate the earlier experiments with i) and ii) VEGF-D and VEGF-C¹⁵⁶ this time with overexpression using adenovirus vectors and iii) PBS. Some cystic mice are injected with adenovirus containing a reporter construct such as β-galactosidase, rather than a VEGF, to assess renal distribution. Groups are assessed at 6, 9 and 12 weeks; good overexpression is expected at 6 weeks, which is expected to fall and be virtually extinguished at 9 and 12 weeks respectively. Mice are left for longer periods if they are cystic but healthy or if they are normal controls. Both are thoroughly examined to rule out tumours or other potential side effects.

Groups treated at 4 weeks: i) Adenovirus overexpression construct VEGF-D, ii) Adenovirus overexpression construct VEGF-C¹⁵⁶, iii) PBS.

3-6 weeks expected duration of VEGF-D or -C¹⁵⁶ overexpression.

Sacrifice 6-8 animals in each group at 6, 9 and 12 weeks with blood and urine collection. Later samples to be collected for survival and side effect analysis from 18 weeks

Experimental end-points: In all experiments, kidneys are harvested with six to eight animals in each group at each time-point. This is an appropriate number of animals for a statistical analysis to be performed with power to demonstrate at least a 50% difference in measured parameters (Chiu et al Am J Pathol 2006 169: 1925-1938; Long et al Kidney Int 2008 74: 300-309).

### REFERENCES

Amura C R, Brodsky K S, Groff R, Gattone V H, Voelker N F, Doctor R B. 2007 VEGF receptor inhibition blocks liver cyst growth in pkd2(WS25/−) mice. Am J Physiol Cell Physiol 293:C419-C428.

Birk D M, Barbato J, Mureebe L, Chaer R A. 2008 Current insights on the biology and clinical aspects of VEGF regulation. Vasc Endovascular Surg 42: 517-530.

Boshart M, Weber F, Jahn G, Dorsch-Hasler K, Fleckenstein B, Schaffner W. (1985) A very strong enhancer is located upstream of an immediate early gene of human cytomegalovirus. Cell 41: 521-530.

Cheetham T D, Holly J M, Clayton K, Cwyfan-Hughes S, Dunger D B. (1995) The effects of repeated daily recombinant human insulin-like growth factor I administration in adolescents with type I diabetes. Diabet Med 12: 885-892.

Chiu M G, Johnson T M, Woolf A S, Dahm-Vicker E M, Long D A, Guay-Woodford L, Hillman K A, Bawumia S, Venner K, Hughes R C, Poirier F, Winyard P J. 2006 Galectin-3 associates with the primary cilium and modulates cyst growth in congenital polycystic kidney disease. Am J PathoL 169: 1925-1938.

Davis H L, Whalen R G, Demeneix B A. 1993 Direct gene transfer into skeletal muscle in vivo: factors affecting efficiency of transfer and stability of expression. Hum Gene Ther. 4 151-159.

Gale N W, Thurston G, Hackett S F, Renard R, Wang Q, McClain J, Martin C, Witte C, Witte M H, Jackson D, Suri C, Campochiaro P A, Wiegand S J, Yancopoulos G D. 2002 Angiopoietin-2 is required for postnatal angiogenesis and lymphatic patterning and only the latter role is rescued by Angiopoietin-1. Dev Cell 3: 411-423.

Gnatenko D, Arnold T E, Zolotukhin S, Nuovo G J, Muzyczka N, Bahou W F. 1997 Characterization of recombinant adeno-associated virus-2 as a vehicle for gene delivery and expression into vascular cells. J Investig Med. 45: 87-98.

Goeters C, Mertes N, Tacke J, Bolder U, Kuhmann M, Lawin P, Dohlein D. 1995 Repeated administration of recombinant human insulin-like growth factor-I in patients after gastric surgery. Effect on metabolic and hormonal patterns. Ann Surg 222: 646-653.

Goldman J, Conley K A, Raehl A, Bondy D M, Pytowski B, Swartz M A, Rutkowski J M, Jaroch D B, Ongstad E L. 2007 Regulation of lymphatic capillary regeneration by interstitial flow in skin. Am J Physiol Heart Circ Physiol 292: H2176-2183.

Gurunluoglu R, Ozer K, Skugor B, Lubiatowski P, Carnevale K, Siemionow M. 2002 Effect of transfection time on the survival of epigastric skin flaps pretreated with adenovirus encoding the VEGF gene. Ann Plast Surg. 49: 161-169.

Huang J L, Woolf A S, Long D A. 2012 Angiogenesis and autosomal dominant polycystic kidney disease. Pediatr Nephroi. September 19. [Epub ahead of print].

Huber S, Bruns C J, Schmid G, Hermann P C, Conrad C, Niess H, Huss R, Graeb C, Jauch K W, Heeschen C, Guba M. 2007 Inhibition of the mammalian target of rapamycin impedes lymphangiogenesis. Kidney Int 71: 771-777.

Jafar T H, Stark P C, Schmid C H, Strandgaard S, Kamper A L, Maschio G, Becker G, Perrone R D, Levey AS; ACE Inhibition in Progressive Renal Disease (AIPRD) Study Group. 2005. The effect of angiotensin-converting-enzyme inhibitors on progression of advanced polycystic kidney disease. Kidney Int 67: 265-271.

Joukov V, Kumar V, Sorsa T, Arighi E, Weich H, Saksela O, Alitalo K. 1998 A recombinant mutant vascular endothelial growth factor-C that has lost vascular endothelial growth factor receptor-2 binding, activation, and vascular permeability activities. J. Biol. Chem. 273: 6599-6602.

Kim V N, Mitrophanous K, Kingsman S M, Kingsman A J. Minimal requirement for a lentivirus vector based on human immunodeficiency virus type 1. J Virol. 72: 811-816.

Korhonen J, Lahtinen I, Halmekytö M, Alhonen L, Janne J, Dumont D, Alitalo K. 1995 Endothelial-specific gene expression directed by the tie gene promoter in vivo. Blood 86: 1828-1835.

Lantinga-van Leeuwen I S, Dauwerse J G, Baelde H J, Leonhard W N, van de Wal A, Ward C J, Verbeek S, Deruiter M C, Breuning M H, De Heer E, Peters D J. 2004. Lowering of Pkd1 expression is sufficient to cause polycystic kidney disease. Hum. Mol. Genet 13: 3069-3077.

Lee A S, Lee J E, Jung Y J, Kim D H, Kang K P, Lee S, Park S K, Lee S Y, Kang M J, Moon W S, Kim H J, Jeong Y B, Sung M J, Kim W. 2013 Vascular endothelial growth factor-C and -D are involved in lymphangiogenesis in mouse unilateral ureteral obstruction. Kidney Int 83: 50-62.

Lehner R, Stamminger T, Mach M. 1991 Comparative sequence analysis of human cytomegalovirus strains. J Clin MicrobioL 29: 2494-2502

Lentine K L, Xiao H, Machnicki G, Gheorghian A, Schnitzler M A. 2010 Renal function and healthcare costs in patients with polycystic kidney disease. Clin J Am Soc Nephroi. 5: 1471-1479.

Lin F J, Chen X, Qin J, Hong Y K, Tsai M J, Tsai S Y. 2010 Direct transcriptional regulation of neuropilin-2 by COUP-TFII modulates multiple steps in murine lymphatic vessel development. J Clin Invest 120: 1694-1707.

Lohela M, Bry M, Tammela T, Alitalo K. 2009 VEGFs and receptors involved in angiogenesis versus lymphangiogenesis. Curr Opin Cell Biol 21:154-165.

Long D A, Price K L, Ioffe E, Gannon C M, Gnudi L, White K E, Yancopoulos G D, Rudge J S, Woolf A S. 2008 Angiopoietin-1 therapy enhances fibrosis and inflammation following folic acid-induced acute renal injury. Kidney Int 74: 300-309.

Peces R, Peces C, Perez-Duenas V, Cuesta-Lopez E, Azorin S, Selgas R. 2009 Rapamycin reduces kidney volume and delays the loss of renal function in a patient with autosomal-dominant polycystic kidney disease. NDT Plus 2: 133-135.

Quantin B, Perricaudet L D, Tajbakhsh S, Mandel J L. 1992 Adenovirus as an expression vector in muscle cells in vivo. Proc Natl Acad Sci USA. 89: 2581-2584.

Rissanen T T, Markkanen J E, Gruchala M, Heikura T, Puranen A, Kettunen M I, Kholova I, Kauppinen R A, Achen M G, Stacker S A, Alitalo K, Yla-Herttuala S. 2003. VEGF-D is the strongest angiogenic and lymphangiogenic effector among VEGFs delivered into skeletal muscle via adenoviruses. Circ. Res., 92: 1098-1106.

Rosenfeld M A, Yoshimura K, Trapnell B C, Yoneyama K, Rosenthal E R, Dalemans W, Fukayama M, Bargon J, Stier L E, Stratford-Perricaudet L, et al. 1992 In vivo transfer of the human cystic fibrosis transmembrane conductance regulator gene to the airway epithelium. Cell. 68: 143-155

Sakamoto I, Ito Y, Mizuno M, Suzuki Y, Sawai A, Tanaka A, Maruyama S, Takei Y, Yuzawa Y, Matsuo S. 2009 Lymphatic vessels develop during tubulointerstitial fibrosis. Kidney Int 75: 828-838.

Stratford-Perricaudet L D, Makeh I, Perricaudet M, Briand P. 1992 Widespread long-term gene transfer to mouse skeletal muscles and heart. J Clin Invest. 90: 626-630.

Suzuki Y, Ito Y, Mizuno M, Kinashi H, Sawai A, Noda Y, Mizuno T, Shimizu H, Fujita Y, Matsui K, Maruyama S, Imai E, Matsuo S, Takei Y. 2012 Transforming growth factor-13 induces vascular endothelial growth factor-C expression leading to lymphangiogenesis in rat unilateral ureteral obstruction. Kidney Int 81: 865-879.

Szuba A, Skobe M, Karkkainen M J, Shin W S, Beynet D P, Rockson N B, Dakhil N, Spilman S, Goris M L, Strauss H W, Quertermous T, Alitalo K, Rockson S G. 2002 Therapeutic lymphangiogenesis with human recombinant VEGF-C. FASEB J 16: 1985-1987.

Tao Y, Kim J, Schrier R W, Edelstein C L. 2005 Rapamycin markedly slows disease progression in a rat model of polycystic kidney disease. J Am Soc Nephrol. 16: 46-51.

Tao Y, Kim J, Yin Y, Zafar I, Falk S, He Z, Faubel S, Schrier R W, Edelstein C L. 2007 VEGF receptor inhibition slows the progression of polycystic kidney disease. Kidney Int 72: 1358-1366.

Torres V E, Chapman A B, Devuyst O, Gansevoort R T, Grantham J J, Higashihara E, Perrone R D, Krasa H B, Ouyang J, Czerwiec F S. 2012 Tolvaptan in Patients with Autosomal Dominant Polycystic Kidney Disease. N. Engl. J. Med. 367: 2407-2418.

Torres V E, Chapman A B, Perrone R D, Bae K T, Abebe K Z, Bost J E, Miskulin D C, Steinman T I, Braun W E, Winklhofer F T, Hogan M C, Oskoui F R, Kelleher C, Masoumi A, Glockner J, Halin N J, Martin D R, Remer E, Patel N, Pedrosa I, Wetzel L H, Thompson P A, Miller J P, Meyers C M, Schrier R W; HALT PKD Study Group. 2012 Analysis of baseline parameters in the HALT polycystic kidney disease trials. Kidney Int 81: 577-585.

Wigle J T, Harvey N, Detmar M, Lagutina I, Grosveld G, Gunn M D, Jackson D G, Oliver G. 2002 An essential role for Prox1 in the induction of the lymphatic endothelial cell phenotype. EMBO J21: 1505-1513.

Wu X, Liu NF. 2011 FOXC2 transcription factor: a novel regulator of lymphangiogenesis. Lymphology 44: 35-41.

Xu Y, Yuan L, Mak J, Pardanaud L, Caunt M, Kasman I, Larrivée B, Del Toro R, Suchting S, Medvinsky A, Silva J, Yang J, Thomas JL, Koch AW, Alitalo K, Eichmann A, Bagri A. 2010. Neuropilin-2 mediates VEGF-C-induced lymphatic sprouting together with VEGFR3. J Cell Biol 188: 115-130.

Yoon Y S, Murayama T, Gravereaux E, Tkebuchava T, Silver M, Curry C, Wecker A, Kirchmair R, Hu C S, Kearney M, Ashare A, Jackson D G, Kubo H, Isner J M, Losordo D W. 2003 VEGF-C gene therapy augments postnatal lymphangiogenesis and ameliorates secondary lymphedema J Clin Invest 111: 717-725.

1.-30. (canceled)

31. A method of treating a renal cystic disease in a subject suffering therefrom, the method comprising administering a compound to the subject,
wherein the compound is a lymphangiogenic agent, or a nucleic acid encoding said agent.

32. The method according to claim 31 wherein the compound is a lymphangiogenic agent which is an agonist of VEGFR-3 or a nucleic acid encoding said agent.

33. A method of treating a renal cystic disease in a subject suffering therefrom, the method comprising administering a compound to the subject,
wherein the compound is an agent selected from the group consisting of:
a VEGF-C polypeptide; a VEGF-D polypeptide; a nucleic acid encoding a VEGF-C polypeptide;
and a nucleic acid encoding a VEGF-D polypeptide.

34. The method according to claim 33, wherein the renal cystic disease is PKD or cystic dysplasia.

35. The method according to claim 34, wherein the PKD is ARPKD or ADPKD.

36. The method according to claim 33, wherein the agent reduces cyst formation or number of cysts in the subject.

37. The method according to claim 33, wherein the agent reduces the size of the disease cysts in the subject.

38. The method according to claim 33, wherein the treatment is to inhibit the progression of the disease, prevent or reduce the likelihood or severity of kidney failure or loss of renal function; or reduce kidney size/body weight ratio.

39. The method according to claim 33, wherein the agent is VEGF-C.

40. The method according to claim 33, wherein the agent is VEGF-D.

41. The method according to claim 33, wherein the agent is a fragment of VEGF-C that retains VEGFR-3 binding and lymphangiogenic activity.

42. The method according to claim 33, wherein the agent is a fragment of VEGF-D that retains VEGFR-3 binding and lymphangiogenic activity.

43. The method according to claim 33, wherein the agent is a variant of VEGF-C sharing at least 70% identity therewith, and that retains VEGFR-3 binding and lymphangiogenic activity.

44. A method according to claim 33, wherein the agent is a variant of VEGF-D sharing at least 70% identity therewith, and that retains VEGFR-3 binding and lymphangiogenic activity.

45. A method according to claim 43, wherein the agent is a derivative of VEGF-C which is VEGF-C¹⁵⁶ where Cys 156 is replaced by a different residue, preferably a Ser residue, and has reduced VEGFR-2 binding affinity relative to VEGF-C.

46. A method according to claim 41 or 42 wherein the agent is a derivative of VEGF-C or VEGF-D which comprises D-amino acids.

47. The method according to claim 31, wherein the agent is a polypeptide produced in the target cells by expression from a heterologous gene encoding the agent introduced into the cells.

48. The method according to claim 33, wherein the compound is a nucleic acid comprising a nucleotide sequence encoding an agent which is a VEGF-C polypeptide or VEGF-D polypeptide.

49. The method according to claim 48, wherein the nucleic acid is a gene therapy vector comprising a promoter operably linked to the nucleotide sequence that encodes the VEGF-C polypeptide or VEGF-D polypeptide for transcription of the sequence that encodes the agent in cells of the mammalian subject.

50. The method according to claim 49, wherein the nucleotide sequence that encodes the VEGF-C polypeptide or VEGF-D polypeptide also encodes a secretory signal sequence in frame with the sequence of the agent.

51. The method according to claim 49, wherein the gene therapy vector is an adenoviral or adeno-associated viral vector.

52. The method according to claim 31, wherein the agent is selected from the list consisting of: angiopoietin-2, coup-tfII, foxc2, neuropilin-2 and prox 1.

53. The method according to claim 33, wherein the compound is administered to said subject in a prophylactically effective amount, for prophylaxis in respect of cyst reduction optionally following diagnosis of the disease or susceptibility to the disease in the subject.

54. The method according to claim 33, wherein the compound is in the form of a composition comprising a compound and a pharmaceutically acceptable carrier or diluent.

55. The method according to claim 33, wherein the compound is administered by injection direct into the target site in the kidney.

56. The method according to claim 55, wherein the compound is administered weekly.

57. The method according to claim 33, wherein the treatment is combined with another therapy for cystic kidney disease, which other therapy is symptomatic or disease modifying.
